(19) United States
(12) Patent Application Publication
(10) Pub. No.: US 2015/0289804 A1
(43) Pub. Date: Oct. 15, 2015

(54) NETWORKED SENSOR SYSTEMS FOR REMOTE PATIENT MONITORING

(71) Applicant: THE REGENTS OF THE UNIVERSITY OF CALIFORNIA, Oakland, CA (US)

(72) Inventor: William J. Kaiser, Los Angeles, CA (US)

(73) Assignee: THE REGENTS OF THE UNIVERSITY OF CALIFORNIA, Oakland, CA (US)

(21) Appl. No.: 14/691,915

(22) Filed: Apr. 21, 2015

Related U.S. Application Data
(63) Continuation of application No. PCT/US2013/064386, filed on Oct. 10, 2013.
(60) Provisional application No. 61/716,837, filed on Oct. 22, 2012.

(51) Int. Cl. A61B 5/00; A61B 5/0476; A61B 5/0488; A61B 5/04; A61B 5/11; A61B 5/145; A61B 5/01; A61B 5/0402; A61B 5/0496

(52) U.S. Cl. CPC A61B 5/4812; A61B 5/0022; A61B 5/0402; A61B 5/0476; A61B 5/0488; A61B 5/0496; A61B 5/11; A61B 5/14542; A61B 5/01; A61B 5/4818; A61B 5/04012; A61B 2505/07

(57) ABSTRACT

A remote patient monitoring system which is particularly well-suited for clinical out of center sleep testing (clinical OCST). Sensor nodes are grouped for wearing on the head, chest, and lower body of the patient. Sensor nodes wirelessly communicate sensor data to a nearby gateway, which communicates this data over a network (e.g., Internet) to one or more servers. The server(s) analyzes the sensor data for usage assurance, and to discern patient sleep patterns and characteristics. Guidance messages are generated by the system based on the analysis. Interfaces are provided to allow the patient to access their own information, and healthcare providers to access information collected regarding their patients.

### CROSS-REFERENCE TO RELATED APPLICATIONS

This application is a 35 U.S.C. §111(a) continuation of PCT international application number PCT/US2013/064386 filed on Oct. 10, 2013, incorporated herein by reference in its entirety, which claims priority to, and the benefit of, U.S. provisional patent application Ser. No. 61/716,837 filed on Oct. 22, 2012, incorporated herein by reference in its entirety. Priority is claimed to each of the foregoing applications.

The above-referenced PCT international application was published as PCT International Publication No. WO 2014/066059 on May 1, 2014, which publication is incorporated by reference herein in its entirety.

### STATEMENT REGARDING FEDERALLY SPONSORED RESEARCH OR DEVELOPMENT

Not Applicable

### INCORPORATION-BY-REFERENCE OF MATERIAL SUBMITTED IN A COMPUTER PROGRAM APPENDIX

Not Applicable

### NOTICE OF MATERIAL SUBJECT TO COPYRIGHT PROTECTION

A portion of the material in this patent document is subject to copyright protection under the copyright laws of the United States and of other countries. The owner of the copyright rights has no objection to the facsimile reproduction by anyone of the patent document or the patent disclosure, as it appears in the United States Patent and Trademark Office publicly available file or records, but otherwise reserves all copyright rights whatsoever. The copyright owner does not hereby waive any of its rights to have this patent document maintained in secrecy, including without limitation its rights pursuant to 37 C.F.R. §1.14.

### BACKGROUND OF THE INVENTION

1. Field of the Invention

This invention pertains generally to remote patient monitoring, and more particularly to out of center sleep testing with a portable sleep testing device.

2. Description of Related Art

It has become widely recognized that sleep quality is critical to the most important health concerns, and that sleep disorders, including sleep apnea, represent large health risks to a significant fraction of the adult population. Driven by multiple needs, the market for sleep monitoring is rapidly emerging, with its need for precise and comprehensive diagnostic information. Currently, clinical sleep monitoring is substantially restricted to testing performed in the clinic or sleep center. It is important to note that clinical monitoring instruments and measurement methods are quite distinct from consumer sleep monitoring devices, which lack a full range of diagnostic capability and accuracy.

Clinical sleep monitoring systems must include a range of precise measurement capabilities in order to meet the requirements for clinical diagnostics, whose use is then reimbursable by a sleep center or healthcare provider. The complete list of sensor systems includes many electrophysiology systems, respiratory measurement systems, motion measurement systems, and others.

Clinical sleep monitoring, as currently performed in a sleep monitoring center (SMC), represents a high cost treatment. Recent regulatory developments have created an acute need for a cost reduced system for remote sleep monitoring (RSM) or out of center sleep testing (OCST) technology which is applicable for operation in residential environments. Regulatory requirements are emerging that require performing OCST operations as a screening step prior to sleep center testing.

The challenges of low cost product development, low cost service, usage assurance, and reliability confront developers of products and services required to meet this need. Currently available technology, having evolved from standard approaches and clinical use, are not directly applicable at low cost to residential usage.

Accordingly, a need exists for low cost OCST system which provides a sufficient range of testing at a sufficient accuracy to be considered a clinical OCST system.

### BRIEF SUMMARY OF THE INVENTION

A remote patient monitoring device is described which is particularly well-suited for use in an out of center sleep testing (OCST) system in the form of a wearable-to-enterprise sleep monitoring (WESM) system. A new next generation OCST architecture is described (OCST-NG), which combines monitoring, data archive and reporting, usage assurance, and subject guidance, and enables significant cost reductions for sleep testing.

Further aspects of the invention will be brought out in the following portions of the specification, wherein the detailed description is for the purpose of fully disclosing preferred embodiments of the invention without placing limitations thereon.

### BRIEF DESCRIPTION OF THE SEVERAL VIEWS OF THE DRAWING(S)

The invention will be more fully understood by reference to the following drawings which are for illustrative purposes only:

FIG. 1 is a block diagram of an out of center sleep testing (OCST) system according to an embodiment of the present invention.

FIG. 2 is a block diagram of a sleep monitoring sensor (SMSN) for use with the out of center sleep testing (OCST) system according to an embodiment of the present invention.

### DETAILED DESCRIPTION OF THE INVENTION

A remote patient monitoring system is described which is particularly well-suited for use in wearable-to-enterprise sleep monitoring (WESM). The system is designed to provide low cost patient monitoring while meeting all requirements for relevant current procedure terminology (CPT) and healthcare common procedure coding system (HCPCS) reimbursement codes.

The WESM incorporates a plurality of sleep monitoring sensors (SMSN) in a new generation of out of center sleep testing which integrates a large number of sensing functions including: (1) electrophysiology sensing, (2) usage assurance sensor functions, (3) interference rejection and noise reduction, and (4) communication systems.

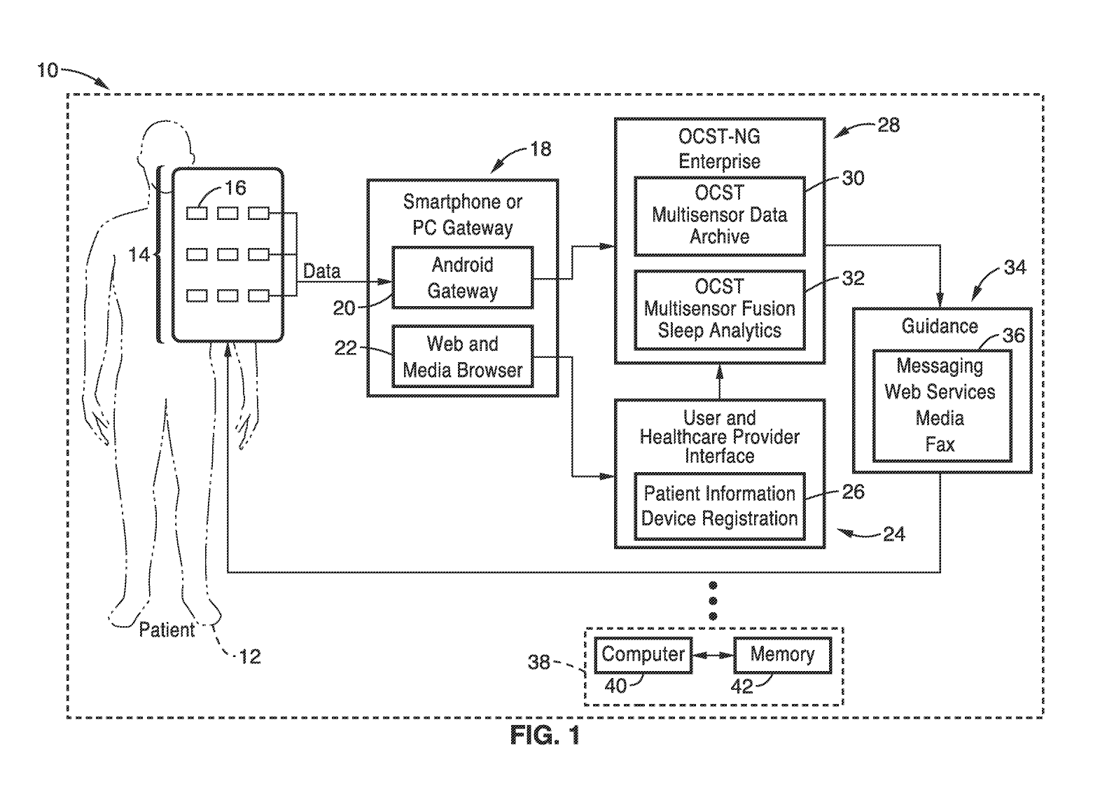

FIG. 1 illustrates an example embodiment 10 of an OCST-NG system. A wearable SMSN array 14 is seen for attachment to a patient 12, with individual SMSN 16, shown grouped (e.g., rows) into wearable head, chest and lower body packages. The SMSN devices 16 communicate with a local gateway 18 that by itself provides network data transport to centralized OCST-NG data archiving and analytics. By way of example and not limitation, the gateway is seen for an Android® device gateway 20 and includes a web and media browser 22, although the gateway can be configured for interfacing with numerous types of cellular phones, portable devices, computers, or other electronic devices.

Through the use of web and media browsers 22, users as well as health-care providers can access patient-specific data via the user and healthcare provider interface 24. This system allows the health care provider to track data collected for their patients, and also provides guidance for users of the OCST-NG system. As shown, the interface 24 contains patient and device registration information 26.

An enterprise level interface also exists 28, which is coupled to the specific gateway (e.g., Android gateway) 20, as well as from interface 24. The enterprise interface 28 collects specific data and performs analysis, such as discerning patient sleep patterns and characteristics, and is shown with an OCST multisensor data archive 30 and OCST multisensor fusion sleep analytics 32. A guidance system 34 is also depicted which communicates with the patients via communication services 36, exemplified as messaging (email and instant messaging), web services, media and FAX, based on the computer analysis of the data collected.

Gateway 18 provides autonomous, stand-alone, network-independent assurance of operation and performs all data acquisition. In addition, gateway 18 provides network access for the enterprise level 28 data collection and analysis. The enterprise level 28 supports remote access, data acquisition and archiving, as well as delivery of data sources to enable physician billing and all other associated requirements. By utilizing the sleep monitoring enterprise system, a competitive advantage is gained by providing for rapid installation, robust data collection and analysis, and communication with patient and healthcare worker interfaces in a system which may be deployed on a large scale.

It should be appreciated that the functions of the system, including collection and analysis of patient sensor data, as well as maintaining the database on patients and devices, and performing automated communication is preferably facilitated utilizing programming executable on one or more computing devices 38, such as containing one or more computers 40 and one or more memories 42. Computational devices are preferably incorporated at the smartphone/PC gateway 18, as well as at the interface level 24, and enterprise level 28, and made use of in the guidance module 34. The gateway device is located nearby the patient as it receives short-range wireless communication from the array of SMSN, while the enterprise level analysis functions and other interfaces are performed by programming executing on one or more servers on a network, preferably the world-wide-web (Internet) configured for communicating with the gateway. It should also be appreciated that control or communication, or a combination of control and communications, within the sleep monitoring sensor node (SMSN) array, or a specific SMSN of that array, can be performed on a computer device 38 (e.g., typically a microcontroller) executing programming for carrying out the control or communication process.

The computational device(s) are configured to generate signals for controlling circuitry, for interfacing with persons, and for collecting, managing and communicating data. It will also be appreciated that the computer readable media (memory) in these computations systems is “non-transitory,” which comprises any and all forms of computer-readable media, with the sole exception being a transitory, propagating signal. Accordingly, the invention may comprise any form of computer-readable media, including those which are random access (e.g., RAM), require periodic refreshing (e.g., DRAM), those that degrade over time (e.g., EEPROMS, disk media), or that store data for only short periods of time and/or only in the presence of power, with the only limitation being that the term “computer readable media” is not applicable to an electronic signal which is transitory.

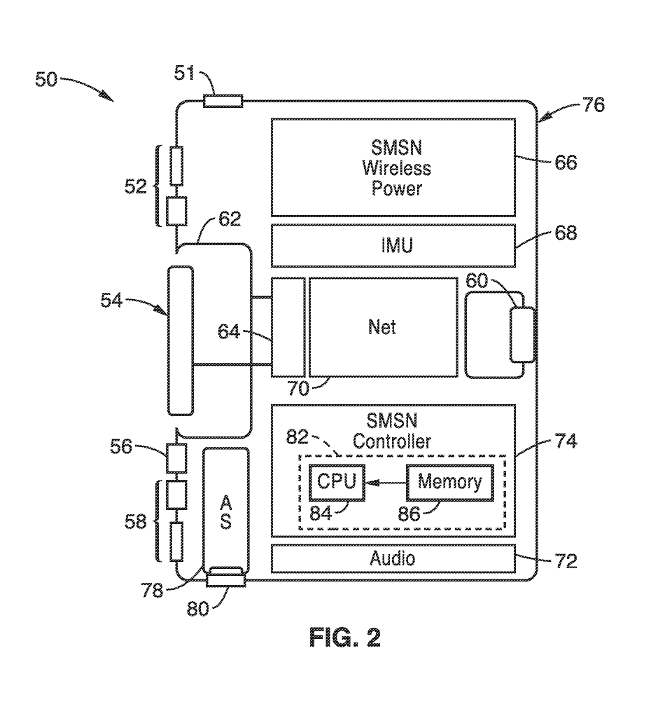

FIG. 2 illustrates an example of an SMSN embodiment 50 integrating the sensor functionality which is wearable by the patient and configured for communication to gateway 18 seen in FIG. 1. The SMSN 50 integrates all electrophysiology, usage assurance, position and motion sensing, and interference rejection and noise reduction functions into a single common unit. Arrays of these sensor nodes (SMSN) are utilized within the OCST-NG system for collecting information on a patient.

The components of the wearable SMSN include elements for wearing on specific parts of the patient's body, such as the head, upper body (chest) and appendages (e.g., legs), and designed for ease-of-use, comfort, appearance, verification, as well as configurability.

In FIG. 2 the SMSN is seen comprising a tissue surface temperature sensor 51, pressure sensor 52, electrophysiology sensor 54 (e.g., EEG, ECG, EOG, EMG electrodes, or the like), blood oxygen sensor 56, pressure sensor 58, and external communications port 60 at the exterior of the SMSN housing 76. Within the device, an active guard shield 62 is seen about (around) the electrophysiology sensor 54, which is coupled to a dry electrode analog processing section 64. Power for the SMSN is derived from a wireless power supply 66. The SMSN unit also provides an integrated motion sensing unit (IMU) 68 which determines subject motion and positioning. A network communications interface 70 is configured for controlling wireless communication with the gateway device 18 in FIG. 1. Inclusion of an audio interface (sensors) 72 is configured to allow audio information to be analyzed, such as sounds associated with certain sleep conditions, including snoring and sleep apnea. The audio information is preferably analyzed in combination with information from the other sensors in determining overall sleep characteristics. Additional sensors are incorporated within additional sensor (AS) area 78 having connector bay 80.

The elements of the SMSN are controlled by controller circuitry 74, such as dedicated electronic circuits, or more preferably at least one computational element 82, such as CPU 84 and memory 86 (e.g., typically a microcontroller or microprocessor and associated memory). Specific elements of the SMSN are described in greater detail in the following sections.

The electroencephalography (EEG) subsystem is integrated with the wearable head unit, and provides a low cost and low operating power solution. The EEG electrodes are developed to enable convenient wearability and reduce interference with hair and to limit electrode number and density. Electrode systems are also configured so as not to detract from personal appearance. The EEG system preferably includes auxiliary sensors that ensure proper usage and compliance and integration with usage guidance.

An electrooculography (EOG) system is integrated with the wearable head unit, as a companion to EEG. The EOG systems was also developed to insure ease-of-use, integral assurance and guidance while not detracting from the user's appearance.

An electrocardiography (ECG) subsystem is preferably integrated within a wearable chest unit. The ECG subsystem takes advantage of a low cost and low operating power solution shared by EEG and EOG systems. The ECG electrodes are configured to enable convenient wearability while reducing dependency on adhesive electrodes. ECG system similarly utilizes auxiliary sensors that ensure proper usage, compliance and integration. The ECG systems also include multipoint measurement inference methods (that do not introduce a cost increase relative to conventional systems) while providing a reliability and effectiveness competitive advantage.

An electromyogram (EMG) subsystem is integrated within a wearable unit including motion sensors that detect assurance of leg installation. The EMG system takes advantage of a low cost and low operating power solution shared by the EEG, EOG, and ECG systems. Similarly, the EMG electrodes are developed to enable convenient wearability and reduce dependency on adhesive electrodes. In addition other ECMG systems include multipoint measurement inference methods.

It should be appreciated that electrophysiological sensor systems are a dominate cost factor in manufacturing OCST products and may also be a primary concern in regard to ease of use, usage assurance, and reliability. The present invention represents a fundamental advance for electrophysiology sensors which is based on a new principle and recent microelectronic advances.

First, the SMSN introduces a new guarded potential measurement method (GPMM) dry electrode system to eliminate the need for expensive wet electrodes and the attendant cost and complexity of their management. In the GPMM approach, dry electrodes from the SMSN are applied to tissue, while the SMSN includes active electrostatic guard methods for assurance of high fidelity coupling. Conventional measurement systems not utilizing these dry sensors, are limited by the inconvenience and short operating lifetime of electrodes that rely on liquid or gel media required to establish low impedance coupling. The SMSN guarded electrode system utilized with the high resolution EEG provides performance equaling conventional wet electrodes in a direct comparison. The SMSN solution also incorporates an auxiliary GPMM unit configured to detect the results of electric field interference and to reduce this at the GPMM sensor system, toward reducing device settling time and increasing operation robustness for subjects exposed to electrical interference.

These electro sensing technologies can be optionally enhanced in a number of different ways. EOG capability can be optionally enhanced with confirmed eye motion detection capability. EEG capability can be optionally enhanced with alpha and beta wave detection along with such phenomena as alpha-wave blocking without requirements for hair removal.

An airflow sensor is preferably integrated with the wearable head unit and supported by sensor sampling systems provided by the SMSN unit. The airflow sensor is implemented within the additional sensors (AS) 78 shown in FIG. 2 with connection 80, and is incorporated within at least one embodiment of the invention. The airflow sensors are configured to provide assurance and compliance monitoring capability. Redundancy is incorporated into the design and the accuracy of airflow determination is increased through utilizing a multipoint measurement that is not sensitive to one or more sensors being occluded. This forms a competitive advantage in cost and performance.

Respiratory effort and rate sensors (RERS) are integrated with a wearable chest unit that is designed as well with integrated usage assurance, compliance, and guidance sensors supported by sensor sampling systems provided by the SMSN unit. RERS is implemented within the additional sensors (AS) 78 shown in FIG. 2 with connection 80, and incorporated within at least one embodiment of the invention. RERS applies both motion and non-contact electrostatic sensing methods and exploits inference methods that combine multiple sources of sensor evidence for determination of respiratory effort and rate.

A sleep time and motion (STM) subsystem is integrated with the EMG subsystem in a wearable unit. The STM system is also preferably configured with a detection method for assurance of leg installation and to guide subject installation accordingly. The STM system also preferably includes use of multipoint measurement inference methods.

A blood oxygenation sensor system (SpO2) 56 is integrated with a wearable chest unit, so that blood oxygenation information can be obtained during testing.

An auditory sensing system 72 is preferably integrated with the wearable chest unit, and is configured for detection of sound emissions associated with conditions, such as snoring or apnea. Classification of snoring level will be included and validated rapidly according to trials. The system provide advantages for performing local classification of snoring or apnea signals and in at least one embodiment avoids the need for recording sound information that may present a potential privacy concern.

An actigraphy sensing subsystem supported by motion sensor and sensor sampling systems is provided by the SMSN unit. The actigraphy sensing subsystem is preferably implemented within the additional sensors (AS) 78 shown in FIG. 2 with connection 80, and is incorporated within at least one embodiment of the invention. Actigraphy sensing detects subject body position, as well as motions associated with restless leg movement (RLM) and periodic leg movement (PLM). In at least one embodiment, this position sensing takes advantage of WHI development for low power, long operating life motion sensing systems that can be worn at the patients ankles. This information is utilized in combination with motion sensing data, such as triaxial accelerometer sensors included in the wearable chest unit. This system provides beneficial local classification of motion and orientation while preferably avoiding any necessary recording of specific location or other information that may present a potential privacy concern.

A user event annotation subsystem is provided in at least one embodiment at the smartphone or PC gateway shown in FIG. 1 (18) which enables individuals wearing the sleep monitoring system, or caregivers, to enter events that facilitate subsequent diagnostic analysis. Event entry preferably includes both a menu-driven interface as well as a free-form event description entry.

The system is also preferably configured to assure user verification and identification at the smartphone or PC gateway shown in FIG. 1 (18). It is recognized that user identity verification will be important for certain applications, and can be performed according to several optional mechanisms. User data entry can include user verification and identification which includes a voice recording of a statement. Sensor signal data analysis indicating proper usage and continued, uninterrupted wearing of components according to schedule, can also ensure that sensor systems have not been removed and reapplied in a manner not compliant with prescription. Still further, in at least one embodiment an optional subject RFID bracelet verification system is integrated to assure proper user identification; such as incorporating an RFID reader coupled to the gateway which is configured for reading RFID information from an RFID bracelet worn by one or more patients.

The SMSN includes motion sensors in each module providing recognition for motion to enable artifact rejection and usage assurance. The motion data from each module is utilized interoperably to determine relative motions of the patient's body and is also a means of cross-checking between sensors to eliminate error.

The SMSN also preferably includes integral tactile pressure sensors 52 and/or 58 in each module to provide assurance of proper application.

Each SMSN device, independent of the area it will be applied to the patient, includes a system board and associated controller as well as other preferably common elements. It should be appreciated that some circuit options on the common system board, may not be fully populated for specific SMSN units not requiring a specific sensor input.

The SMSN common system board (e.g., printed circuit board) preferably includes: (a) GPMM sensor electrodes with GPMM analog interface; (b) GPMM interference detection and rejection circuitry; (c) SpO2 blood oxygen sensor interface; (d) an in-contact tissue temperature sensor; (e) module temperature sensor; (f) application force pressure sensors; (g) acceleration and rotation motion and orientation sensors (IMU); (h) audio sensing system; (i) analog sensor sampling; (j) microcontroller signal acquisition and processing; (k) short-distance (e.g., bluetooth) wireless interface; (l) high efficiency long life power source, such as LiPoly battery, and smart battery management; and (m) wireless power recharging.

The SMSN wearable systems and its integrated sensors provide multiple advantages, including the following. The OCST-NG wearable systems include SMSN modules integrated with elastomer systems designed for comfort in sleep and ease of use. The OCST-NG respiratory effort sensor is preferably included in the chest unit SMSN, which directly measures thorax motion through an articulated system exploiting low cost conductive, elastic fabric. The OCST-NG airflow sensor is similarly configured for comfort, ease of use, and data redundancy. The SMSN provides a data interface to the OSCT-NG gateway.

Beneficial OCST-NG gateway functionality preferably includes the following. Processing and storage of EEG, ECG, EOG, and EMG signals utilizing biomedical device signal processing, sensor fusion, as well as interference detection and rejection. SpO2 blood oxygenation computation. Usage assurance computation and determination through tracking of time history of applied force and sensor signals in application of tactile pressure sensors. Motion artifact computation and determination through motion sensor data stream computation. Subject orientation through motion sensor data stream computation processing and storage of audio signals using acoustic biomedical device signal processing and sensor fusion for multiple applications.

Embodiments of the present invention may be described with reference to flowchart illustrations of methods and systems according to embodiments of the invention, and/or algorithms, formulae, or other computational depictions, which may also be implemented as computer program products. In this regard, each block or step of a flowchart, and combinations of blocks (and/or steps) in a flowchart, algorithm, formula, or computational depiction can be implemented by various means, such as hardware, firmware, and/or software including one or more computer program instructions embodied in computer-readable program code logic. As will be appreciated, any such computer program instructions may be loaded onto a computer, including without limitation a general purpose computer or special purpose computer, or other programmable processing apparatus to produce a machine, such that the computer program instructions which execute on the computer or other programmable processing apparatus create means for implementing the functions specified in the block(s) of the flowchart(s).

Accordingly, blocks of the flowcharts, algorithms, formulae, or computational depictions support combinations of means for performing the specified functions, combinations of steps for performing the specified functions, and computer program instructions, such as embodied in computer-readable program code logic means, for performing the specified functions. It will also be understood that each block of the flowchart illustrations, algorithms, formulae, or computational depictions and combinations thereof described herein, can be implemented by special purpose hardware-based computer systems which perform the specified functions or steps, or combinations of special purpose hardware and computer-readable program code logic means.

Furthermore, these computer program instructions, such as embodied in computer-readable program code logic, may also be stored in a computer-readable memory that can direct a computer or other programmable processing apparatus to function in a particular manner, such that the instructions stored in the computer-readable memory produce an article of manufacture including instruction means which implement the function specified in the block(s) of the flowchart(s). The computer program instructions may also be loaded onto a computer or other programmable processing apparatus to cause a series of operational steps to be performed on the computer or other programmable processing apparatus to produce a computer-implemented process such that the instructions which execute on the computer or other programmable processing apparatus provide steps for implementing the functions specified in the block(s) of the flowchart(s), algorithm(s), formula(e), or computational depiction(s).

From the discussion above it will be appreciated that the invention can be embodied in various ways, including the following:

1. An apparatus for remote patient monitoring, comprising: an array of patient monitoring sensor nodes configured for wearing by a patient, each patient monitoring sensor node configured for wirelessly communicating sensor data; a gateway device configured for wirelessly receiving sensor data from said array of patient monitoring sensor nodes; at least one server configured for connection to said gateway; at least one processor on said at least one server; and programming executable on said processor for processing patient sensor data, said processing comprising: analyzing sensor data collected from said array of patient monitoring sensor nodes and storing results in a patient database; and generating guidance messages to the patient based on said results.

2. The apparatus of any of the previous embodiments, wherein said sensor nodes are grouped for wearing on different areas of a patient's body.

3. The apparatus of any of the previous embodiments, wherein said sensor nodes are grouped into packages for wearing on head, chest, and lower body of a patient.

4. The apparatus of any of the previous embodiments, wherein said programming is configured for analyzing said sensor data for usage assurance.

5. The apparatus of any of the previous embodiments, wherein said remote patient monitoring apparatus is a portable sleep testing device configured for performing out of center clinical sleep testing (clinical OCST).

6. The apparatus of any of the previous embodiments, wherein said programming is configured for analyzing said sensor data to discern patient sleep patterns and characteristics.

7. The apparatus of any of the previous embodiments, wherein said sensors comprise electrophysiology sensing, position and motion sensing, and usage assurance sensing.

8. The apparatus of any of the previous embodiments, wherein said electrophysiology sensing is selected from the group of electrophysiology sensor types consisting of EEG, ECG, EOG and EMG.

9. The apparatus of any of the previous embodiments, further comprising an active guard shield around said electrophysiology sensor, said guard shield coupled to a dry electrode analog processing section.

10. The apparatus of any of the previous embodiments, wherein said sensors further comprise blood oxygen level sensing, tissue surface temperature sensing, pressure sensing.

11. The apparatus of any of the previous embodiments, wherein said sensors further comprise an audio interface configured for detecting sounds associated with certain sleep conditions.

12. The apparatus recited in claim 11, wherein said audio interface is configured for detecting sounds associated with snoring, sleep apnea, or a combination of snoring and sleep apnea.

13. The apparatus of any of the previous embodiments, wherein said programming generates said guidance messages to the patient using forms of electronic communication selected from the group of electronic communication forms consisting of electronic mail, instant messaging, web services, media and FAX.

14. The apparatus of any of the previous embodiments, wherein said programming is further configured for providing the patient and health-care providers access to patient-specific data through a user interface on said at least one server.

15. The apparatus of any of the previous embodiments, wherein said programming is configured for maintaining a database of patient and remote patient monitoring apparatus registration information.

16. An apparatus for out of center clinical sleep testing, comprising: an array of sleep monitoring sensor nodes (SMSN) configured for wearing by a patient, each patient monitoring sensor node configured for wirelessly communicating sensor data; wherein said sensor nodes are grouped for wearing on head, chest, and lower body of the patient; wherein said sensors on said sensor nodes comprise electrophysiology sensing, position and motion sensing, and usage assurance sensors; a gateway device configured for wirelessly receiving sensor data from said array of patient monitoring sensor nodes; at least one server configured for connection to said gateway; at least one processor on said at least one server; and programming executable on said processor for processing patient sensor data, said processing comprising: analyzing said sensor data for usage assurance; analyzing sensor data collected from said array of sleep monitoring sensor nodes to discern patient sleep patterns and characteristics and storing results in a patient database; and generating guidance messages to the patient based on said results.

17. The apparatus of any of the previous embodiments, wherein said electrophysiology sensing is selected from the group of electrophysiology sensor types consisting of EEG, ECG, EOG and EMG; and wherein said electrophysiology sensing incorporates an active guard shield coupled to a dry electrode analog processing section.

18. The apparatus of any of the previous embodiments, wherein said sensors further comprise an audio interface configured for detecting sounds associated with certain sleep conditions.

19. The apparatus of any of the previous embodiments, wherein said audio interface is configured for detecting sounds associated with snoring, sleep apnea, or a combination of snoring and sleep apnea.

20. An apparatus for out of center clinical sleep testing, comprising: an array of sleep monitoring sensor nodes (SMSN) configured for wearing by a patient, each patient monitoring sensor node configured for wirelessly communicating sensor data; wherein said sensor nodes are grouped for wearing on head, chest, and lower body of the patient; wherein said sensors on said sensor nodes comprise electrophysiology sensing, position and motion sensing, usage assurance sensors, and audio sensing; wherein said electrophysiology sensing is selected from the group of electrophysiology sensor types consisting of EEG, ECG, EOG and EMG; wherein said electrophysiology sensing incorporates an active guard shield coupled to a dry electrode analog processing section; wherein said audio sensing is configured for detecting sounds associated with snoring, sleep apnea, or a combination of snoring and sleep apnea; a gateway device configured for wirelessly receiving sensor data from said array of patient monitoring sensor nodes; at least one server configured for connection to said gateway; at least one processor on said at least one server; and programming executable on said processor for processing patient sensor data, said processing comprising: analyzing said sensor data for usage assurance; analyzing said sensor data for sounds and other characteristics associated with snoring, sleep apnea, or a combination of snoring and sleep apnea; analyzing sensor data collected from said array of sleep monitoring sensor nodes to discern patient sleep patterns and characteristics and storing results in a patient database; and generating guidance messages to the patient based on said results.

Although the description herein contains many details, these should not be construed as limiting the scope of the disclosure but as merely providing illustrations of some of the presently preferred embodiments. Therefore, it will be appreciated that the scope of the disclosure fully encompasses other embodiments which may become obvious to those skilled in the art.

In the claims, reference to an element in the singular is not intended to mean “one and only one” unless explicitly so stated, but rather “one or more.” All structural, chemical, and functional equivalents to the elements of the disclosed embodiments that are known to those of ordinary skill in the art are expressly incorporated herein by reference and are intended to be encompassed by the present claims. Furthermore, no element, component, or method step in the present disclosure is intended to be dedicated to the public regardless of whether the element, component, or method step is explicitly recited in the claims. No claim element herein is to be construed as a “means plus function” element unless the element is expressly recited using the phrase “means for”. No claim element herein is to be construed as a “step plus function” element unless the element is expressly recited using the phrase “step for”.

What is claimed is:

1. An apparatus for remote patient monitoring, the apparatus comprising:
an array of patient monitoring sensor nodes configured for wearing by a patient, each patient monitoring sensor node configured for wirelessly communicating sensor data;
a gateway device configured for wirelessly receiving sensor data from said array of patient monitoring sensor nodes;
at least one server configured for connection to said gateway;
at least one processor on said at least one server; and
programming executable on said processor for processing patient sensor data, said processing comprising:
analyzing sensor data collected from said array of patient monitoring sensor nodes and storing results in a patient database; and
generating guidance messages to the patient based on said results.

2. The apparatus recited in claim 1, wherein said sensor nodes are grouped for wearing on different areas of a patient's body.

3. The apparatus recited in claim 2, wherein said sensor nodes are grouped into packages for wearing on head, chest, and lower body of a patient.

4. The apparatus recited in claim 1, wherein said programming is configured for analyzing said sensor data for usage assurance.

5. The apparatus recited in claim 1, wherein said remote patient monitoring apparatus is a portable sleep testing device configured for performing out of center clinical sleep testing (clinical OCST).

6. The apparatus recited in claim 5, wherein said programming is configured for analyzing said sensor data to discern patient sleep patterns and characteristics.

7. The apparatus recited in claim 5, wherein said sensors comprise electrophysiology sensing, position and motion sensing, and usage assurance sensing.

8. The apparatus recited in claim 7, wherein said electrophysiology sensing is selected from the group of electrophysiology sensor types consisting of EEG, ECG, EOG and EMG.

9. The apparatus recited in claim 8, further comprising an active guard shield around said electrophysiology sensor, said guard shield coupled to a dry electrode analog processing section.

10. The apparatus recited in claim 7, wherein said sensors further comprise blood oxygen level sensing, tissue surface temperature sensing, and pressure sensing.

11. The apparatus recited in claim 7, wherein said sensors further comprise an audio interface configured for detecting sounds associated with certain sleep conditions.

12. The apparatus recited in claim 11, wherein said audio interface is configured for detecting sounds associated with snoring, sleep apnea, or a combination of snoring and sleep apnea.

13. The apparatus recited in claim 1, wherein said programming generates said guidance messages to the patient using forms of electronic communication selected from the group of electronic communication forms consisting of electronic mail, instant messaging, web services, media and FAX.

14. The apparatus recited in claim 1, wherein said programming is further configured for providing the patient and health-care providers access to patient-specific data through a user interface on said at least one server.

15. The apparatus recited in claim 1, wherein said programming is configured for maintaining a database of patient and remote patient monitoring apparatus registration information.

16. An apparatus for out of center clinical sleep testing, comprising:
an array of sleep monitoring sensor nodes (SMSN) configured for wearing by a patient, each patient monitoring sensor node configured for wirelessly communicating sensor data;
wherein said sensor nodes are grouped for wearing on head, chest, and lower body of the patient; and
wherein said sensors on said sensor nodes comprise electrophysiology sensing, position and motion sensing, and usage assurance sensors;
a gateway device configured for wirelessly receiving sensor data from said array of patient monitoring sensor nodes;
at least one server configured for connection to said gateway;
at least one processor on said at least one server; and
programming executable on said processor for processing patient sensor data, said processing comprising:
analyzing said sensor data for usage assurance;
analyzing sensor data collected from said array of sleep monitoring sensor nodes to discern patient sleep patterns and characteristics and storing results in a patient database; and
generating guidance messages to the patient based on said results.

17. The apparatus recited in claim 16:
wherein said electrophysiology sensing is selected from the group of electrophysiology sensor types consisting of EEG, ECG, EOG and EMG; and
wherein said electrophysiology sensing incorporates an active guard shield coupled to a dry electrode analog processing section.

18. The apparatus recited in claim 16, wherein said sensors further comprise an audio interface configured for detecting sounds associated with certain sleep conditions.

19. The apparatus recited in claim 18, wherein said audio interface is configured for detecting sounds associated with snoring, sleep apnea, or a combination of snoring and sleep apnea.

20. An apparatus for out of center clinical sleep testing, comprising:
an array of sleep monitoring sensor nodes (SMSN) configured for wearing by a patient, each patient monitoring sensor node configured for wirelessly communicating sensor data;
wherein said sensor nodes are grouped for wearing on head, chest, and lower body of the patient;
wherein said sensors on said sensor nodes comprise electrophysiology sensing, position and motion sensing, usage assurance sensors, and audio sensing;
wherein said electrophysiology sensing is selected from the group of electrophysiology sensor types consisting of EEG, ECG, EOG and EMG;
wherein said electrophysiology sensing incorporates an active guard shield coupled to a dry electrode analog processing section; and
wherein said audio sensing is configured for detecting sounds associated with snoring, sleep apnea, or a combination of snoring and sleep apnea;
a gateway device configured for wirelessly receiving sensor data from said array of patient monitoring sensor nodes;
at least one server configured for connection to said gateway;
at least one processor on said at least one server; and
programming executable on said processor for processing patient sensor data, said processing comprising:
analyzing said sensor data for usage assurance;
analyzing said sensor data for sounds and other characteristics associated with snoring, sleep apnea, or a combination of snoring and sleep apnea;
analyzing sensor data collected from said array of sleep monitoring sensor nodes to discern patient sleep patterns and characteristics and storing results in a patient database; and
generating guidance messages to the patient based on said results.
